(19) United States
(12) Patent Application Publication
(10) Pub. No.: US 2009/0105526 A1
(43) Pub. Date: Apr. 23, 2009

(54) Surgical procedure for correcting cystocele and rectocele

(76) Inventors: Donato Piroli Torelli, Napoli (IT); Mario Polichetti, Roccapiemonte (IT)

Correspondence Address: MODIANO & ASSOCIATI, Via Meravigli, 16, Milano, 20123, IT

(21) Appl. No.: 12/285,582

(22) Filed: Oct. 9, 2008

(30) Foreign Application Priority Data
Oct. 17, 2007 (IT) MI2007A002011

(51) Int. Cl. A61F 2/02

(52) U.S. Cl. 600/30

(57) ABSTRACT

A surgical procedure for correcting cystocele, comprising the steps of:
identifying a center of the cystocele;
performing an infiltration of the cystocele with physiological solution;
performing a longitudinal incision;
identifying the pubocervical fascia and performing a longitudinal incision therein;
defining four paravesical tunnels directed toward the upper and lower poles of the obturator foramina bilaterally;
introducing a mesh with four articulated arms, each arm being introduced in a corresponding tunnel;
suturing the longitudinal incision.

The present invention relates to a surgical procedure for correcting cystocele and rectocele.

### BACKGROUND OF THE INVENTION

Cystocele is a hernial disorder of the bladder, whereas rectocele is a hernial disorder of the rectum. The term “hernia” refers to the displacement of an organ out of its natural location.

As is known, currently correction of anterior colpocele or cystocele can be performed with a traditional colpoplasty technique or by means of a colpoplasty operation with application of a prosthesis.

In the case of correction of anterior colpocele or cystocele, the procedure requires an initial disinfection of the vagina, an application of a bladder catheter, the placement of vaginal valves and the visualization of the operating field. At this point, a clamping of the neck of the uterus is performed and the uterus is pulled down/outwardly, and an infiltration with physiological solution, with a vasoconstrictor, of the vaginal wall is performed.

The surgeon then performs an incision of the anterior vaginal wall in a longitudinal direction from the vesicouterine plica (neck of the uterus) to the region of the neck of the bladder (urethra) for approximately ⅞ centimeters up to 10/12 centimeters, thus performing a broad parting of the vagina from the bladder.

The surgical procedure then provides for a reduction of bladder volume with purse-string or silk stitches or fascial stitches or by applying a prosthesis and fixing it by means of separate stitches.

The excess vaginal flaps are then cut and extended from the prolapsed condition. These can be very large flaps.

Finally, suture of the vagina is provided, and the catheter is left in place for three days, performing bladder gymnastics for 1/3-4 days and with vaginal tamponade for 1-2 days.

The surgical technique for the operation for correcting posterior colpocele or rectocele is substantially similar to the one described earlier, with the difference that the incision step is a transverse incision of the vulvar entrance and a subsequent longitudinal incision of the vaginal wall affected by the rectovaginal prolapse.

This step is followed by a parting of the vaginal wall from the rectum, with extensive resection of the excess vaginal flaps. Subsequently, suture of the fascia of the rectum is provided by means of separate stitches and by means of the mutual approach of the fibers of the central region of the levator ani muscles or by means of the application of a prosthesis and its fixation by means of separate stitches.

The techniques described above, which are substantially similar as regards correction of cystocele and correction of rectocele, are however affected by drawbacks linked to the fact that the procedures are invasive for the patient, with long times for healing and resumption of normal everyday's activity.

### SUMMARY OF THE INVENTION

The aim of the present invention is to provide a surgical procedure for correcting cystocele and rectocele that has minimal invasiveness with respect to corresponding known types of operations.

Within this aim, an object of the present invention is to provide a surgical procedure for correcting cystocele and rectocele that allows to provide an incision of the anterior vaginal wall or of the vulvar entrance that is distinctly smaller than the incisions performed with known types of surgical procedures.

Another object of the present invention is to provide a surgical procedure for correcting cystocele and rectocele that allows to have an extremely rapid and less painful postoperative course than corresponding known types of operations.

Still another object of the present invention is to provide a surgical procedure for correcting rectocele and cystocele that is highly reliable and relatively simple to provide and requires a reduced amount of time.

This aim, as well as these and other objects that will become better apparent hereinafter, are achieved by a surgical procedure for correcting cystocele, characterized in that it comprises the steps of:

identifying the center of the cystocele;

performing an infiltration of the cystocele with physiological solution;

performing a longitudinal incision;

identifying the pubocervical fascia and performing a longitudinal incision therein;

defining four paravesical tunnels directed toward the upper and lower poles of the obturator foramina bilaterally;

introducing a mesh with four articulated arms, each arm being introduced in a corresponding tunnel;

suturing the longitudinal incision.

This aim and these objects are also achieved by a surgical procedure for correcting rectocele, characterized in that it comprises the steps of:

pinching the posterior vaginal wall;

pinching the perineum, creating a vaginal plica;

providing a longitudinal incision of the vagina;

parting at least partially the rectum from the vagina;

defining, by means of a tunneling instrument, two pararectal recesses starting from the upper corner of the pinched vagina;

arranging a mesh with two articulated arms in said pararectal recesses;

suturing the vaginal wall.

### BRIEF DESCRIPTION OF THE DRAWINGS

Further characteristics and advantages of the invention will become better apparent from the description of preferred but not exclusive embodiments of the procedure according to the present invention, illustrated by way of non-limiting example in the accompanying drawings, wherein:

FIG. 1 is a view of the step of preparation for the cystocele correction operation;

FIG. 2 is a view of a second step of the cystocele correction operation;

FIG. 3 is a view of a third step of cystocele correction;

FIG. 4 is a view of a first step for the rectocele correction operation;

FIG. 5 is a view of a second step of the rectocele correction operation;

FIG. 6 is a view of a third step of the rectocele correction operation;

FIG. 7 is a view of a variation of the rectocele correction operation.

### DESCRIPTION OF THE PREFERRED EMBODIMENTS

With reference to the figures, the cystocele correction operation is described first, followed by the rectocele correction operation, the two operations, i.e., the two surgical procedures, being substantially similar.

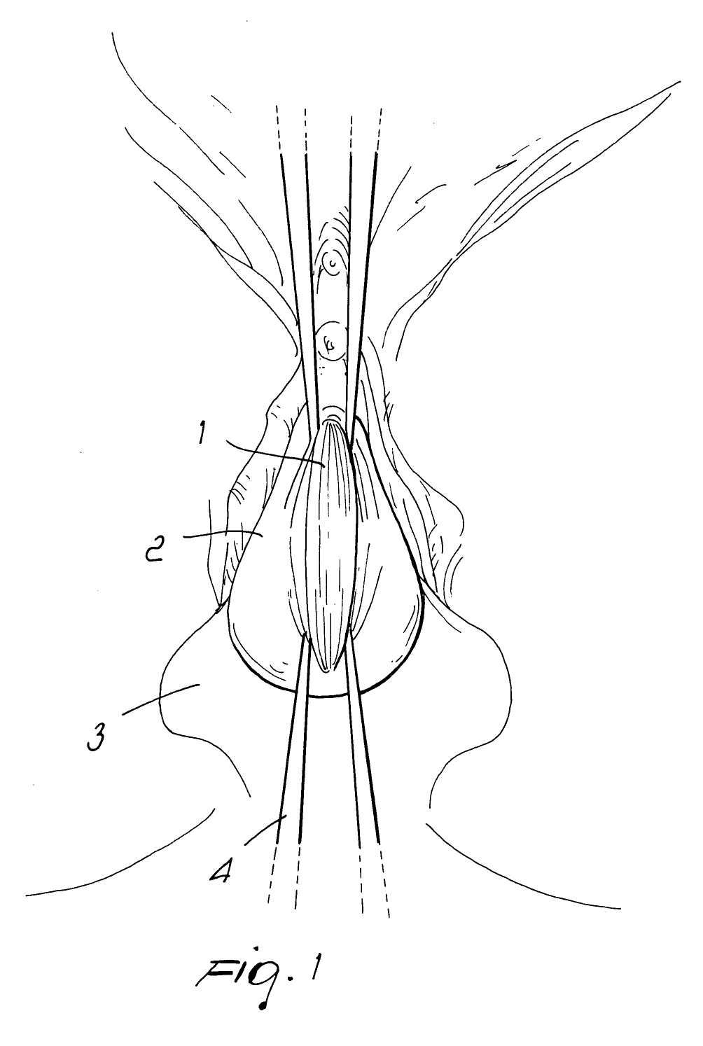

As regards the cystocele correction operation, and therefore with reference to FIGS. 1 to 3, the surgical procedure provides for a first step for disinfecting and delimiting the operating field with sterile drapes, followed by a step for catheterization of the bladder.

At this point, the cystocele is identified and its extension is assessed, as shown in FIG. 1. In FIG. 1, the reference numeral 1 designates the region of incision of the vagina, the reference numeral 2 designates the anterior vaginal wall, the reference numeral 3 designates a valve or instrument that is adapted to lower the posterior longitudinal wall.

The reference numeral 4 instead designates Allis forceps.

Therefore, in order to facilitate identification of the cystocele and assess its extension, the valve 3 is used and is inserted appropriately in the vagina. Once the center of the cystocele, designated by the reference numeral 1, has been identified, the anterior wall 2 of the vagina is pinched with two Allis forceps 4 arranged so that they are longitudinally mutually spaced by approximately 2.5 centimeters, taking care to include between them the central part of the cystocele, again as shown in FIG. 1. At this point, an infiltration of the part with physiological solution is performed in order to facilitate the subsequent parting of the tissues.

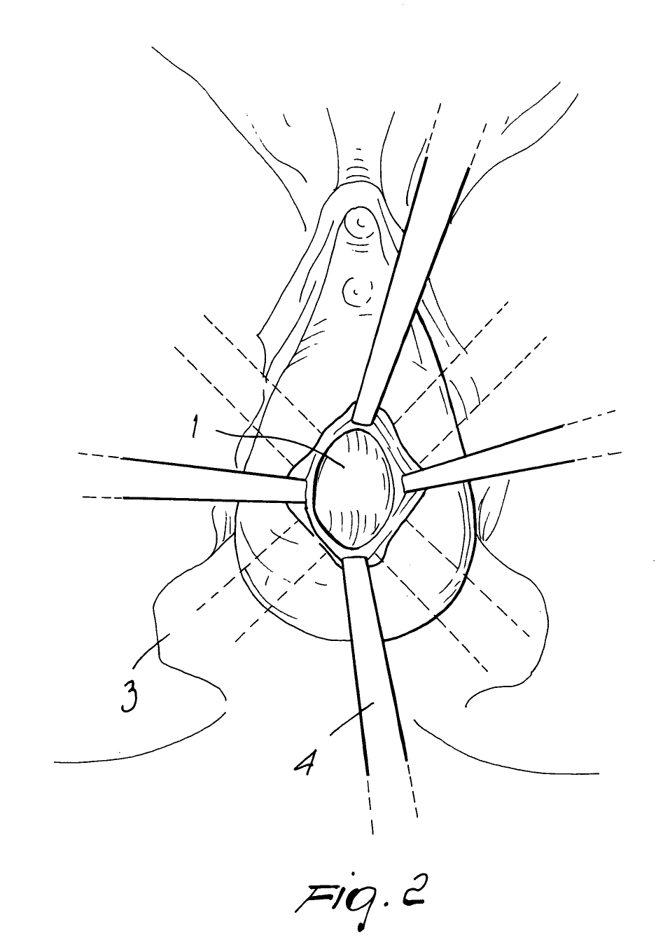

The surgeon, while gripping the two Allis forceps 4 in order to apply a slight traction, produces with the scalpel a small longitudinal incision of approximately 1.5-2 cm. At this point, the lateral margins of the incision are everted as shown in FIG. 2 by means of two additional Allis forceps, applying a slight traction that divaricates them. After identifying the pubocervical fascia, it is cut longitudinally, again for no more than 2 cm.

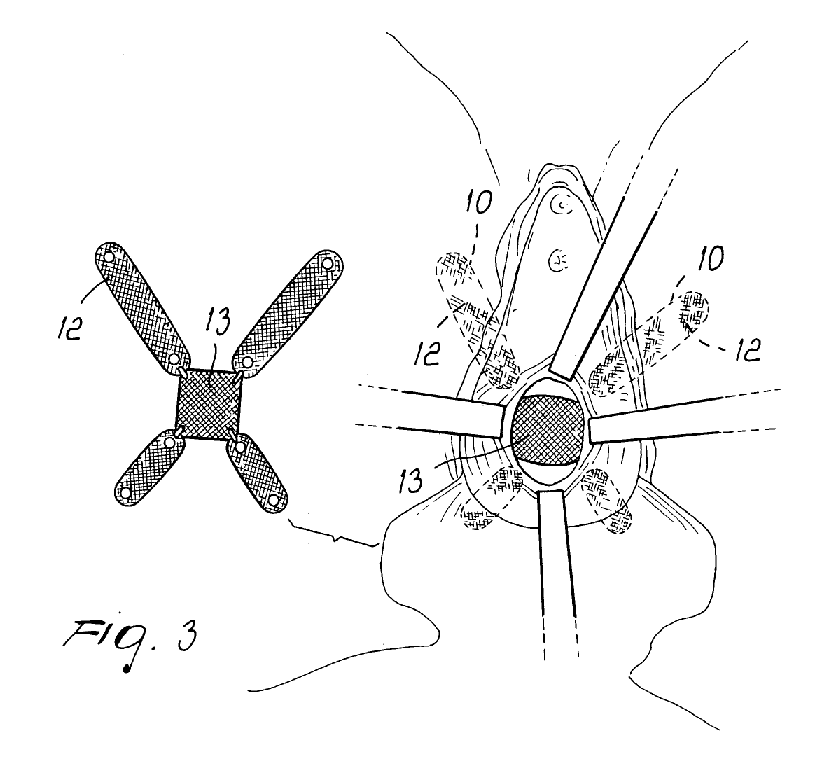

At this point, four paravesical tunnels, directed toward the upper and lower poles of the obturator foramina bilaterally, are provided by means of a tunneling instrument. FIG. 3 is a view of the provision of the tunnels, where the reference numeral 10 designates the tunnels provided by means of the tunneling instrument. Conveniently, the tunneling instrument is provided with a prehensile end, by means of which the four articulated arms of a polypropylene mesh 11 are engaged one by one and the arms 12 of the mesh are pushed into the tunnels 10 prepared previously, so that the central part 13 of the mesh adheres uniformly to the cystocele. The upper tunnels are provided longer than the lower tunnels, as shown also in FIG. 3, and therefore the anterior articulated arms of the mesh are longer than the posterior arms. At the end of the procedure, the small opening or incision is sutured with a double layer.

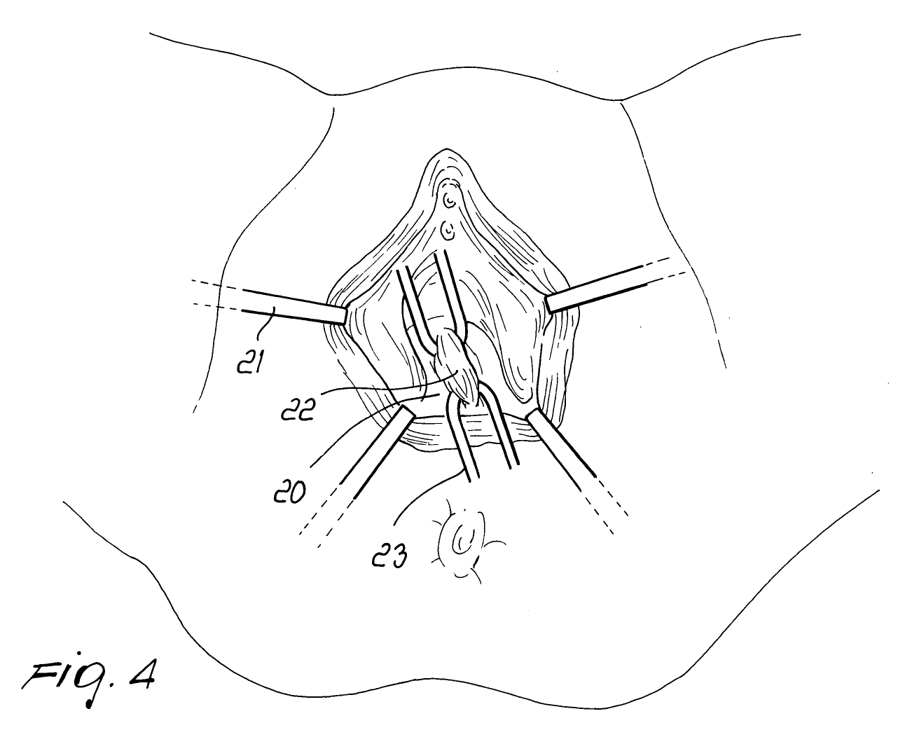

The surgical procedure for correcting rectocele instead provides for the following steps, with reference to FIGS. 4 to 7.

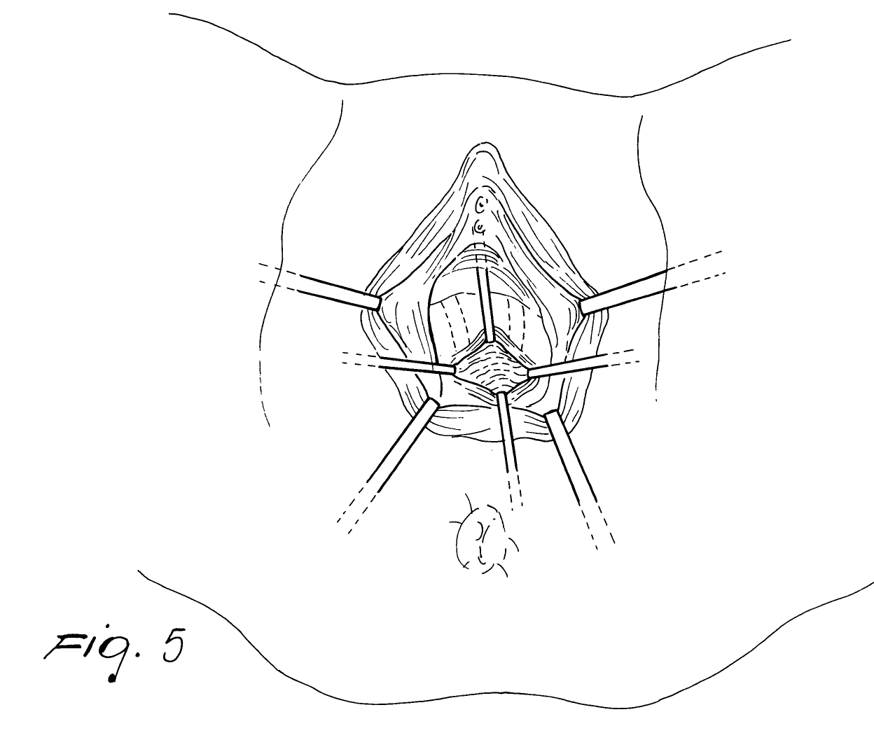

The surgical procedure provides for pinching the posterior vaginal wall approximately 3 centimeters from the perineal margin centrally with Allis forceps. The reference numeral 20 designates, in FIG. 4, the posterior vaginal wall, while the reference numeral 21 designates the Allis forceps. The perineum is then pinched centrally, creating a vaginal plica between the two Allis forceps, with the reference numeral 22 designating the plica. An infiltration with physiological solution is performed at the center of the plica 22 and an incision is provided in the vagina longitudinally. The lateral margins of the incision are then pinched with two additional Allis forceps 23, as shown in FIGS. 4 and 5, and by applying a slight traction, the rectum is partially and delicately parted from the vagina.

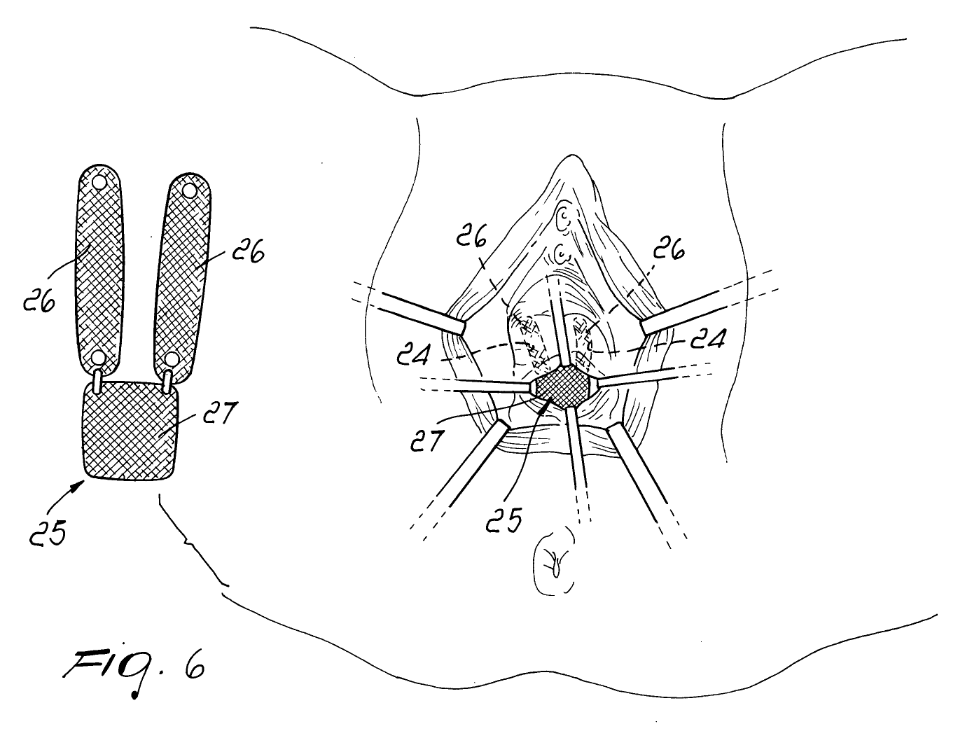

At this point, by using the tunneling instrument described above, two pararectal recesses are provided starting from the upper corner of the pinched vagina, as shown in FIG. 6. In this figure, the pararectal recesses are designated by the reference numeral 24.

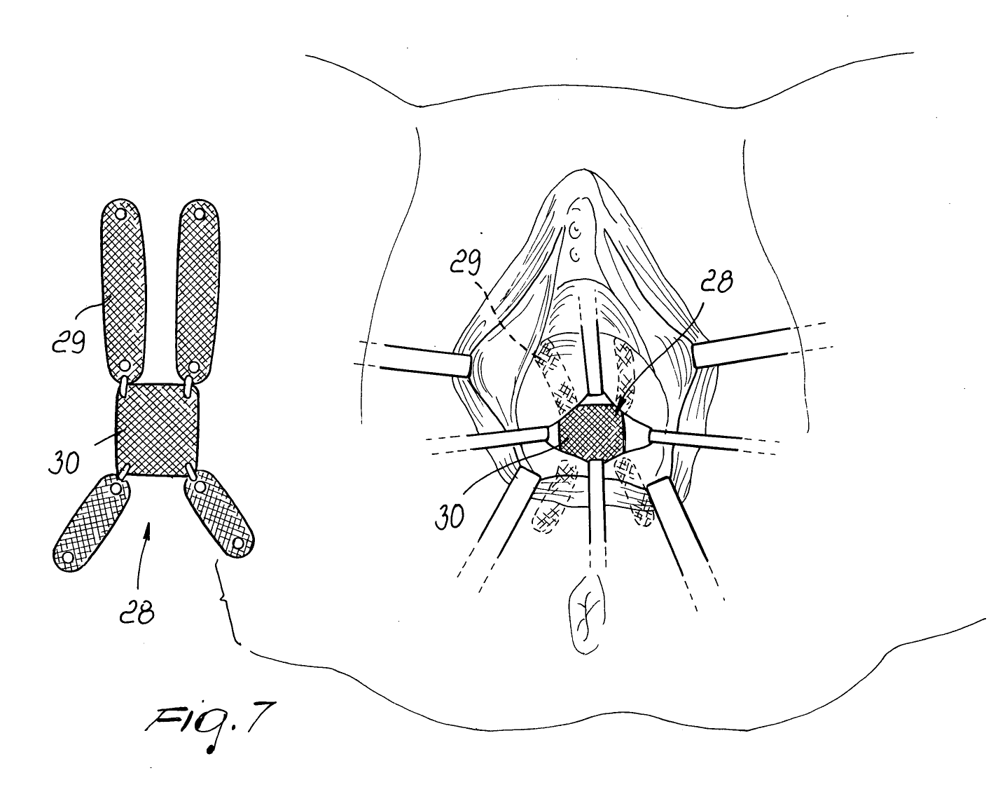

Using the prehensile portion of the tunneling instrument, a mesh 25 is then positioned which is provided with at least two articulated arms 26. The arms 26 are then pushed into the previously created recesses, so that the central portion 27 of the mesh arranges itself uniformly in the rectovaginal space, as shown in FIG. 6. If deemed necessary, one uses a mesh 28 provided with four articulated arms 29, as shown in FIG. 7, which is thus inserted with its central region 30 as defined previously, but in this case four pararectal tunnels are provided in order to accommodate the four arms 29.

Substantially, in addition to the two pararectal tunnels, two tunnels are also provided which are directed toward the levator ani muscles. The mesh is introduced first posteriorly and then anteriorly in the prepared recesses, making sure that the central part 30 arranges itself correctly in the rectovaginal space. At this point, suture of the vaginal wall is performed.

The surgical procedures defined above to correct rectocele and cystocele allow to perform a far less invasive operation than the techniques currently in use, with benefit for the patient, who has far shorter recovery times.

The procedure thus conceived is susceptible of numerous modifications and variations, all of which are within the scope of the appended claims; all the details may further be replaced with other technically equivalent elements.

The disclosures in Italian Patent Application no. MI2007A002011, from which this application claims priority, are incorporated herein by reference.

What is claimed is:

1. A surgical procedure for correcting cystocele, comprising the steps of:
identifying a center of the cystocele;
performing an infiltration of said cystocele with physiological solution;
performing a longitudinal incision;
identifying a pubocervical fascia and performing a longitudinal incision therein;
defining four paravesical tunnels directed toward upper and lower poles of obturator foramina bilaterally;
introducing a mesh with four articulated arms, each arm being introduced in a corresponding tunnel;
suturing said longitudinal incision.

2. The procedure according to claim 1, comprising an initial step for introducing a rear valve in the vagina.

3. The procedure according to claim 1, comprising a step of pinching an anterior wall of the vagina after identifying the center of the cystocele.

4. The procedure according to claim 1, wherein said mesh has a central part that is adapted to adhere uniformly to the cystocele.

5. The procedure according to claim 1, wherein said mesh has two arms articulated along two short arms suitable respectively to be inserted in said tunnels.

6. A surgical procedure for correcting rectocele, comprising the steps of:
pinching a posterior vaginal wall;
pinching the perineum, creating a vaginal plica;
providing a longitudinal incision of the vagina;
parting at least partially the rectum from the vagina;
defining, by means of a tunneling instrument, two pararectal recesses starting from the upper corner of the pinched vagina;
arranging a mesh with two articulated arms in the pararectal recesses;
suturing the vaginal wall.

7. The procedure according to claim 6, comprising a step of infiltrating physiological solution at a center of said plica.

8. The procedure according to claim 6, comprising a step of pinching lateral margins of the provided incision and, by applying a slight traction, parting the rectum from the vagina.

9. The procedure according to claim 6, wherein said mesh comprises two additional articulated arms adapted to be inserted in two additional tunnels adapted to be provided toward the levator ani muscles.
